(19) United States
(12) Patent Application Publication
(10) Pub. No.: US 2026/0199195 A1
(43) Pub. Date: Jul. 16, 2026

(54) ZINC SULFIDE INTERFERENCE PIGMENTS, SYNTHESIS METHOD AND COSMETIC COMPOSITION

(71) Applicants: LVMH RECHERCHE, Saint-Jean-de-Braye (FR); UGIEL, Portets (FR)

(72) Inventors: Guillaume SALEK, Pessac (FR); Aurélie LE BEULZE, Fargues-Saint-Hilaire (FR); Jérôme MAJIMEL, Portets (FR); Emilie GOMBART DE MATOS, Saint-Jean-de-Braye (FR); Valérie ALARD, Saint-Jean-de-Braye (FR)

(21) Appl. No.: 19/135,639

(22) PCT Filed: Dec. 5, 2023
(86) PCT No.: PCT/FR2023/051933

§ 371 Date: Jun. 4, 2025

(30) Foreign Application Priority Data
Dec. 5, 2022 (FR) FR2212769
Dec. 5, 2022 (FR) FR2212775
Dec. 5, 2022 (FR) FR2212777

(51) Int. Cl. A61K 8/02; A61K 8/25; A61K 8/27; A61K 8/28; A61Q 1/02

(52) U.S. Cl. CPC A61K 8/0266; A61K 8/25; A61K 8/27; A61K 8/28; A61Q 1/02; A61K 2800/436

(57) ABSTRACT

The present invention relates to interference pigments comprising a layer of a zinc sulfide material. The pigments advantageously comprise a low-refractive-index core and at least one high-refractive-index layer comprising zinc sulfide. The invention also proposes a method for synthesizing these pigments by heterogeneous precipitation of zinc sulfide on a pulverulent support, the precipitation being obtained by titration of a sodium sulfide solution at a fixed pH, during the addition of a zinc saline solution in a reaction medium containing a dispersion of the pulverulent support, at moderate temperature.

The invention also relates to cosmetic compositions comprising these interference pigments which are free of titanium dioxide, wherein the titanium dioxide is generally introduced as a filler or by providing a cosmetic ingredient containing it.

### TECHNICAL FIELD

The present invention relates to interference pigments comprising at least one zinc sulfide layer which may be free of titanium dioxide.

The invention also relates to a method for synthesizing these pigments, and cosmetic compositions containing them, these compositions themselves being able to be free of titanium dioxide or containing very small amounts thereof.

### PRIOR ART

Generally, white interference effect pigments are obtained by depositing a layer of a high-refractive-index material on the surface of semi-transparent platelet particles of mica, glass or alumina of lower refractive index.

The most commonly used high-index material is titanium dioxide, and there remains a need for alternative pigments that comprise a high-refractive-index material to generate physical colors.

### DISCLOSURE OF THE INVENTION

The present invention addresses this need and provides zinc sulfide pigments, a compound having the advantage of being suitable for a wide variety of industrial applications. The proposed pigments comprise a core covered with a thin layer of zinc sulfide.

The invention provides in particular interference pigments comprising a low-refractive-index core and at least one layer of a zinc sulfide compound whose thickness is chosen to generate silvery white or colored reflections by interference.

The invention also proposes a method for the liquid deposition of submicron layers of a zinc sulfide (ZnS) compound on a micrometric support. The synthesis method can advantageously follow a heterogeneous nucleation and homogeneous growth mechanism, which results in the deposition of a zinc sulfide layer whose chemical composition, crystallinity, crystallography, density and thickness can be controlled. It is unexpected, under mild chemistry conditions and in a liquid process, to succeed in depositing on a pulverulent support such as mica for example, a homogeneous zinc sulfide layer, of controlled thickness, crystallinity, crystallography and chemical composition. This method allows to manufacture zinc sulfide pigments using a method carried out in water, at moderate temperature and at moderate pH.

The interference pigment of the present invention comprises a support and at least one layer of a material comprising high-refractive-index zinc sulfide.

According to a first variant, the pigment is a multi-layer interference pigment lacking a layer containing titanium dioxide.

The pigment may comprise titanium dioxide, but it is preferred that it contains less than 0.1% by mass relative to the mass of the pigment.

The invention advantageously allows to partially or completely replace the titanium dioxide used for the manufacture of interference pigments and allows to offer cosmetic products with low titanium dioxide contents.

In a second variant of the invention, the pigment is an interference, single-layer or multi-layer pigment the support of which comprises a gas or consists of a gas such as air.

### BRIEF DESCRIPTION OF THE DRAWINGS

FIG. 1 is an elementary EDX map of a pigment according to the invention.

FIG. 2 is a scanning electron microscopy image of a pigment according to the invention.

FIG. 3 is an X-ray diffractogram of a pigment according to the invention.

FIG. 4 is a transmission electron microscopy image of a pigment according to the invention comprising an outer protective layer of silica.

FIG. 5 shows the characterization by scanning electron microscopy of gypsum platelet supports used for the preparation of the hollow core of a pigment of the invention.

FIG. 6 and FIG. 7 show the scanning electron microscopy characterization of gypsum platelets covered with a silica layer at 2 different magnifications.

FIG. 8 is an elementary characterization by EDX analysis of gypsum platelet powder covered with a silica layer.

FIG. 9 and FIG. 10 show the characterizations by transmission electron microscopy after dissolution of the gypsum platelets covered with a layer of silica at two different magnifications.

FIG. 11 and FIG. 12 show the characterizations by scanning electron microscopy after dissolution of gypsum platelets covered with a layer of silica at two different magnifications.

FIG. 13 shows the elemental characterization by EDX analysis on silica capsule powder after dissolution of the gypsum.

### DESCRIPTION OF THE EMBODIMENTS

A first object of the invention relates to an interference single-layer or multi-layer pigment, comprising a core and at least one layer of a material comprising zinc sulfide.

The pigment of the invention may comprise a core whose refractive index preferably ranges from 1.00 to 2.10, covered with, or delimited by, at least one layer of a material comprising zinc sulfide.

The layer comprising zinc sulfide preferably has an average physical thickness ranging from 10 nm to 350 nm.

The average physical thickness of the layer of a material comprising zinc sulfide may range, for example, from 20 nm to 340 nm, from 30 nm to 330 nm, from 40 nm to 320 nm, from 50 nm to 310 nm, from 60 nm to 300 nm, from 70 nm to 290 nm, from 80 nm to 280 nm, from 90 nm to 270 nm, from 100 nm to 260 nm or from 110 nm to 250 nm. The physical thickness, also called optical thickness, may be measured by any method known to the person skilled in the art.

The zinc sulfide may represent between 1% and 100% by mass of the mass of the pigment, and the interference pigment of the invention advantageously comprises between 1% and 70% by mass of zinc sulfide relative to the mass of the pigment, advantageously from 15% to 65% by mass, more advantageously from 20% to 60% by mass, even more preferably from 25% to 55% by mass of the mass of the pigment.

In a particularly preferred embodiment, the zinc sulfide represents from 30% to 60% by mass, preferably from 30% to 50% by mass and even better from 35% to 45% by mass of the mass of the pigment.

The pigment may lack an interference layer containing titanium dioxide.

In a particularly advantageous embodiment of the invention, the overall composition of the interference pigment of the invention, including the core and the layer of the material comprising zinc sulfide, is free of titanium dioxide in the sense that it contains less than 10% by mass, preferably less than 5% by mass, or even less than 1% by mass, relative to the mass of the interference pigment. The pigment of the invention.

The interference pigment of the invention is advantageously a powder consisting of particles each of which comprises a core covered with at least one layer of a material comprising zinc sulfide.

Its dimension is a number-average dimension of a population of particles, advantageously equal to the D50 of the population of particles constituting the pigment. In the case of the present invention, the value of the D50 is calculated from the particle size distributions obtained by conventional methods such as laser diffraction or image analysis obtained by scanning electron microscopy (SEM) or by transmission electron microscope (TEM).

The pigment may thus have a dimension, the dimension being able to be defined as D50, ranging from 1 micron to 2000 microns, preferably ranging from 10 microns to 500 microns, while the core may have a dimension ranging from 1 micron to 2000 microns, preferably from 10 microns to 500 microns.

The pigment of the invention is an interference pigment that can be colored or white. This pigment can also generate one or more reflections of different colors, the color of the reflection(s) being able to be different from the color of the pigment itself. The reflections can thus be silvery white or another color. In the cosmetic field, for example, a distinction is made between white mother-of-pearls with silvery white reflections, white mother-of-pearls with colored reflections, colored mother-of-pearls with silvery white reflections and colored mother-of-pearls with colored reflections. These reflections can result in pearly and/or iridescent effects during the transmission and reflection of light through them, caused by optical interference phenomena. The interference color generated results from the strengthening or destruction of the rays of light reflected according to certain wavelengths. A layer of the material comprising zinc sulfide deposited on a support with a lower refractive index can generate destructive and constructive waves that themselves generate a color. Destructive interference of a given wavelength occurs if the reflections from the two surfaces air/material and material/support are completely out of phase. For example, a minimum reflection occurs for incident light perpendicular to a wavelength λ for the layer of the material comprising zinc sulfide, with refractive index N and thickness e, when N*e=(n−1)*λ/2, where n is an integer. In the case where the layer of the material is illuminated by white light, all wavelengths except A appear in the reflection. Enhancement of a given wavelength occurs if the reflections from the two surfaces of the layer of material comprising zinc sulfide are in phase with each other. Thus, for incident light perpendicular to the free surface of the layer, this occurs when N*e=(2n−1)*λ/4.

The layer of material comprising zinc sulfide is preferably an interference layer. For the purposes of the invention, the term “interference layer” means a layer of a material whose optical thickness is capable of generating an optical color when it is deposited on a given support or on a layer of another material.

The material comprising zinc sulfide preferably consists predominantly of zinc sulfide. “Consisting predominantly of zinc sulfide” means a material comprising more than 50% by mass of zinc sulfide relative to the mass of the material.

In one embodiment, the material comprising zinc sulfide consists essentially of zinc sulfide.

The term “consisting essentially of zinc sulfide” means a material comprising at least 90% by mass of zinc sulfide, preferably at least 99% by mass of zinc sulfide relative to the mass of said material.

The material comprising zinc sulfide is a solid solution of zinc sulfide, that is to say a material forming a single crystalline phase comprising zinc sulfide. The person skilled in the art will be able to characterize the crystalline phase number of the material, for example by X-ray diffraction.

The material comprising zinc sulfide may be zinc sulfide called “doped” zinc sulfide, that is to say a material essentially consisting of zinc sulfide and at least one chemical element, the chemical element preferably being in the form of a metal ion.

Thus, in another embodiment, the material comprising sulfide is a material essentially consisting of zinc sulfide and at least one metal ion selected from Fe²⁺, Cu²⁺, Mn²⁺, Ag⁺, Au³⁺, Eu³⁺, Al³⁺, Ce³⁺ and In³⁺.

“Essentially consisting of zinc sulfide and at least one metal ion” means a material comprising at least 90% by mass, preferably at least 99% by mass, of a mixture of zinc sulfide and metal ion(s) relative to the mass of the material. In this embodiment, the chemical element, which is preferably a metal ion, is advantageously present in an amount such that it forms a solid solution, that is to say a single crystalline phase, with the zinc sulfide.

The layer of a material comprising zinc sulfide preferably has a refractive index ranging from 2.30 to 2.90, for example from 2.35 to 2.80, from 2.40 to 2.70, from 2.45 to 2.65, from 2.50 to 2.50. The refractive index of the layer of a material comprising zinc sulfide is preferably close to 2.40.

The pigment of the invention may comprise an outer protective layer. This outer protective layer is advantageously transparent and without a coloring function, so that it is different from an interference layer comprising a zinc sulfide material. The outer protective layer may be obtained by a surface treatment with a chemical compound fulfilling a function of interest. The protective layer may, for example, facilitate the formulation of the pigment in solvents or oils, or protect against UV rays.

The outer protective layer may be organic or mineral in nature, and hydrophilic or hydrophobic in nature. Said layer is for example an inorganic layer such as silica or ceria or a polymer such as PMMA, polystyrene or polyvinyl chloride.

The pigment of the invention may advantageously undergo an additional treatment of a hydrophobic nature, in order to facilitate the dispersion of said pigment in a fatty phase, for example an oily phase. Such a treatment aims at applying a surface agent of a hydrophobic nature to all or part of the surface of the pigment. Such a surface agent may advantageously be selected from amino acids, metal soaps, esters, silicone or fluorinated compounds, acrylic compounds, or else lipids, or a mixture of at least two of these compounds.

The amino acid surfactant may be, for example, glycine, alanine, sarcosine, proline, hydroxyproline, aspartic acid, glutamic acid or lysine, or a derivative such as, for example, an acylated amino acid comprising a saturated or unsaturated fatty acid comprising from 1 to 22 carbon atoms, preferably from 8 to 20 carbon atoms. Such an acylated amino acid surfactant may be, for example, stearoyl glutamic acid, lauroyl glutamic acid, lauroyl aspartic acid, myristoyl glutamic acid, stearoyl lysine, lauroyl lysine, myristoyl lysine and palmitoyl proline or a salt thereof such as a sodium, potassium, calcium, magnesium or aluminum salt. Particularly preferred agents in salt form are preferably sodium myristoyl glutamate, disodium stearoyl glutamate, sodium lauroyl aspartate, dilauramido-glutamide lysine, sodium palmitoyl sarcosine, magnesium palmitoyl glutamate and disodium cocoyl glutamate.

As a surfactant of the metal soap type, aluminum myristate and magnesium stearate may advantageously be mentioned.

As ester-type surfactants, advantageous mention may be made of isostearyl sebacate, dextrin and fatty acid esters such as dextrin stearate, dextrin isostearate, dextrin palmitate, or polyglyceryl-2 tetraisostearate.

As silicone surfactant, mention may advantageously be made of methicone, hydrogenodimethicone, dimethicone, tetramethoxysilane, methyltrimethoxysilane, dimethyldimethoxysilane, tetraethoxysilane, methyltriethoxysilane, dimethyldiethoxysilane, isobutyltrimethoxysilane, decyltrimethoxysilane, octyltrimethoxysilane, octyltriethoxysilane, octadecyltriethoxysilane and hexadecyltriethoxysilane.

As fluorinated surfactant, mention may advantageously be made of perfluorohexylethyltriethoxysilane, trifluoropropyltrimethoxysilane, trifluoropropyltriethoxysilane, tridecafluorooctyltrimethoxysilane or tridecafluorooctyltriethoxysilane.

As acrylic-type surfactants, mention may be made of (co)polymers comprising ethyl(meth)acrylate, butyl(meth)acrylate, octyl(meth)acrylate, decyl(meth)acrylate, lauryl(meth)acrylate, myristyl(meth)acrylate, palmityl(meth)acrylate, stearyl(meth)acrylate, oleyl(meth)acrylate or else linoleyl(meth)acrylate groups.

Examples of lipid surfactants include phospholipids, lecithin, triglycerides, fats, oils, waxes, such agents advantageously being of natural origin. The outer protective layer may also comprise a UV-blocking material such as cerium-doped silica. A UV-protective layer has the advantage of stabilizing the color and/or reflections of the pigment over time, particularly throughout the lifetime of use of the formulations and manufactured products comprising the pigment.

The pigment of the invention may be single-layered, in the sense that it comprises a single layer covering the core, or multi-layered.

According to a first embodiment, the pigment is a single-layer pigment comprising a single layer of a material comprising zinc sulfide. “A single layer” means a layer of homogeneous composition, which can be applied in one or more manufacturing method steps, preferably in a single step. The pigment of the invention can alternatively be multi-layered, that is to say comprise at least two layers of different composition, including at least one layer of a material comprising zinc sulfide and one layer not comprising zinc sulfide. Preferably, the core does not comprise several layer(s) of different materials, and its composition is homogeneous.

The pigment of the invention may comprise, independently of the number of layers of different materials which may cover the core, one or more layers of a material comprising zinc sulfide.

The pigment may comprise, for example, two layers of a material containing zinc sulfide, a first layer consisting of zinc sulfide and a second layer consisting of doped zinc sulfide, as described above, separated from each other by an intermediate layer of low-refractive-index, comprising, for example, silica.

In addition to the material comprising zinc sulfide, the pigment may comprise at least one material whose refractive index ranges from 2.30 to 2.90, such as for example a material selected from the group consisting of Fe₂O₃, FeTiO₃, Cr₂O₃ and/or Fe₃O₄. This material may be part of the composition of the material of the zinc sulfide layer or be in the form of a layer separate from the zinc layer. Thus, the layer comprising zinc sulfide may comprise, in addition to zinc sulfide, at least one material whose refractive index ranges from 2.30 to 2.90. According to a variant, the pigment comprises a first layer consisting of zinc sulfide which is covered and in contact with a second layer consisting of a material different from zinc sulfide whose refractive index ranges from 2.30 to 2.90.

In a second particular embodiment of the invention, the interference pigment is multi-layered and comprises at least one alternation of a first layer of a material comprising zinc sulfide having a refractive index ranging from 2.30 to 2.90, and a second layer, contiguous to the first, of a material having a refractive index ranging from 1.00 to 2.10, the difference in refractive index between said first layer and said second contiguous layer being greater than or equal to 0.3, and preferably ranging from 0.3 to 1.5, more preferably being of the order of 1.3.

In this description, “of the order of” or “close to” means a value that is equal to plus or minus 10% of the given numerical value, or a value taking into account measurement uncertainties.

The refractive index of the layer of a material whose refractive index ranges from 1.00 to 2.10, preferably ranges from 1.00 to 1.60.

In the interference pigment of the invention, the layer of a material comprising zinc sulfide may be covered on one of its surfaces with a continuous or semi-continuous layer of metal nanoparticles, for example gold, silver or copper nanoparticles, the nanoparticles having for example at least one average dimension less than or equal to 500 nm, preferably less than or equal to 100 nm.

The addition of metal nanoparticles can generate a black background and a more or less significant color path of the mother-of-pearl. The metal nanoparticles can be nanoparticles of noble metals such as gold or silver, or nanoparticles of copper or chromium, which are preferably deposited in a semi-continuous manner. Depending on the content of precious metals deposited on the final layer of the pigment, which has a high-refractive-index, such as ZnS, or a low-refractive-index, such as silica, a black background can be generated, which allows in particular white mother-of-pearls with reflections to have a core color close to their reflection color depending on the amount of nanoparticles deposited. The white mother-of-pearl with reflections becomes a colored mother-of-pearl with colored reflections. In addition, adding nanoparticles having absorption in the visible will modify to a greater or lesser extent the reflection and transmission color of the mother-of-pearl.

A layer of silicon dioxide with a thickness ranging from 1 nm to 1 micron can be intercalated between the core and the zinc sulfide layer, and be in contact with both the core and the zinc sulfide layer. It can also be post-treated to generate porosity within the silica layer.

A multi-layer interference pigment of the invention may comprise a stack of several layers comprising at least one layer comprising zinc sulfide having a refractive index ranging from 2.30 to 2.90, at least one layer of a material having a refractive index ranging from 1.00 to 2.10, and at least one layer of a material not comprising zinc sulfide and having a refractive index ranging from 2.30 to 2.90, the order and thickness of the different layers being selected to generate colored reflections.

The pigment of the invention can be synthesized by physical or chemical deposition of zinc sulfide on a solid support.

In the case of physical deposition, the layer of material comprising zinc sulfide can be deposited on the solid support by an atomic layer deposition method.

In the case of chemical deposition, the pigment can be obtained by heterogeneous precipitation of zinc sulfide on the support, from a zinc saline solution and a sodium sulfide solution. It can alternatively be synthesized by bubbling gaseous H₂S or by thermal decomposition of a sulfur precursor as a substitute for sodium sulfide, in the presence of the support. For example, a semi-transparent powdery support such as mica is used, but any type of support, such as one of the solid substrates described previously in the context of the description of the pigment of the invention, is suitable.

Another method for synthesizing the pigment of the invention uses a homogeneous precipitation method. This synthesis method consists of slowly releasing one of the sulfur-containing reactants by raising the temperature during the reaction. The released reactant is distributed homogeneously throughout the volume of the solution, which allows to maintain a low and uniform supersaturation over time. This allows to generate a layer of zinc sulfide on a solid support in a controlled manner. According to this method, a suspension of solid particles, a zinc salt and a precipitating agent that is inactive at room temperature are mixed. The precipitating agent, which allows to generate hydrogen sulfide by thermal decomposition, is for example thioacetamide using water as the reaction solvent, or thiourea by preferentially choosing a heavy alcohol such as a polyol as the solvent.

In one embodiment, the heterogeneous precipitation of zinc sulfide can be carried out by titration of a sodium sulfide solution at a fixed pH, in the reaction medium comprising the solid particles and a zinc saline solution in dilute medium. The sodium sulfide solution is added continuously and the amount is regulated by a titrator according to a pH fixed at the start. The objective is to be at all times in low supersaturation conditions which allow the heterogeneous germination of the zinc sulfide on the solid particles and the formation of a continuous and homogeneous layer of zinc sulfide on their surface. The titrator containing the sodium sulfide solution is used to compensate for the decrease in pH during the addition of the zinc saline solution by maintaining a pre-determined fixed pH, called stationary pH. The stationary pH is chosen so as to form a continuous, homogeneous, compact layer of controlled thickness of zinc sulfide on the solid particles. Depending on the amount of zinc salt added, the thickness of the zinc sulfide layer increases until it reaches the optimal physical thickness conditions for generating interference colors.

A second object of the invention relates to a method for synthesizing a pigment, for example an interference pigment, which consists of the heterogeneous precipitation of a material comprising zinc sulfide onto solid particles. The material comprising zinc sulfide may be zinc sulfide or a solid solution of zinc sulfide and a metal ion as described above.

This method may comprise:
a first step of preparing an aqueous dispersion of solid particles, the dispersion having a pH ranging from 2 to 8 and a temperature close to boiling, and
a second step of coating the solid particles comprising the addition, to said aqueous dispersion, of an aqueous solution of a zinc salt, such as zinc nitrate, and of an aqueous solution of sodium sulfide, the pH of the reaction medium obtained being maintained between 2 and 7 to obtain a suspension of particles covered with the material comprising zinc sulfide.

The homogeneity of the zinc sulfide layer deposited on the surface of the particles can be controlled by different physicochemical parameters such as temperature, concentration of the saline solution and pH.

The concentration of the solid particle dispersion that was prepared in the first step ranges, for example, from 1 g/L to 20 g/L or from 5 g/L to 15 g/L.

The second step is a step of coating the solid particles which is preferably carried out while stirring the reaction medium. The temperature of the reaction medium during the coating step can range from 20° C. to 100° C., preferably from 60° C. to 90° C.

The solid particles may be selected from natural micas, synthetic micas, alkaline earth carbonates such as calcium carbonate, alkaline earth sulfates such as barium sulfate, natural pearls such as guanine or hypoxanthine, an alumina, aluminum, a silica, a borosilicate, a perlite, an organic polymer (such as a plastic), a metal oxide such as zinc oxide or bismuth oxychloride. Their size may range from 1 micron to 2000 microns, preferably from 10 microns to 500 microns. The size is defined in the same way as the size of the substrate described above in the context of the first object of the invention and may be measured by scanning electron microscope (SEM) or by transmission electron microscope (TEM). The particles are preferably platelet-shaped, but may be in any other shape.

Concerning the aqueous saline zinc solution, it is preferably diluted and acidified. Its concentration can range from 0.001 M to 10 M or from 0.01 M to 0.1 M, and its addition rate in the aqueous dispersion of solid particles can range from 0.001 mL/min to 10 mL/min, or from 0.1 mL/min to 1 mL/min. The zinc salt is preferably zinc nitrate.

The aqueous sodium sulfide solution is a dilute solution which can have a concentration between 0.5 M and 3 M. The pH of the reaction medium is preferably maintained between 2 and 7 throughout the synthesis by controlled addition of the dilute sodium sulfide solution.

The method of the invention may comprise a third step, which is a washing and drying step consisting of centrifuging the suspension of the zinc sulfide-covered particles, redispersing the particles in ethanol, then drying them at a temperature ranging from 20° C. to 80° C. for 12 to 24 hours.

It may be necessary to carry out a step of annealing the particles covered with zinc sulfide so as to densify the zinc sulfide layer and intensify the color of the interference pigment. The method of the invention may therefore comprise a step during which the particles covered with zinc sulfide obtained at the end of the third coating step or the fourth washing and drying step as described above, undergo a heat treatment. The particles covered with zinc sulfide may be placed, under air or under argon, at a temperature which may reach the thermal decomposition temperature of ZnS. The synthesis method of the invention may comprise a step of coating the particles covered with zinc sulfide with an organic or mineral matrix in order to form a protective layer. This will be referred to interchangeably as coating or encapsulation.

This encapsulation step, when it consists of a coating with silica, can be carried out by a sol-gel method known to the person skilled in the art. According to a variant of this method, the particles covered with zinc sulfide which have been dried are dispersed in a citric acid solution brought to a base pH. This solution is injected into a water-ethanol reaction medium (for example 25/75 v/v). A silica precursor, for example tetraethyl orthosilicate (abbreviated TEOS), is then added to the mixture, and the solution is left stirring for 24 hours at room temperature. The suspension is then centrifuged, washed with ethanol then dried for 24 hours in an oven at 80° C.

Finally, the synthesis method of the invention may comprise a step of total or partial removal of the core of a support consisting of solid particles, so as to obtain a solid porous core, or a gaseous core. Once the layer of material comprising the zinc sulfide has been deposited on the solid particles at the end of the second step, the removal of the core may be carried out by chemical or physical means. For example, it is possible to use calcium carbonate particles, cover them with zinc sulfide, then dissolve the calcium carbonate with an acid without damaging the structure of the layer of material comprising the zinc sulfide.

The core of the pigment preferably has a refractive index less than or equal to 2.20, preferably ranging from 1.00 to 2.10. It is made up of one or more materials each having a refractive index less than or equal to 2.20, preferably ranging from 1.00 to 2.10, the refractive index being able to be measured by any method known to the person skilled in the art.

The chemical nature of the pigment core can vary. The core can be a gas, such as air, a solid substrate that can be porous or non-porous, or it can comprise both a gas and a solid. The solid substrate is, for example, a platelet-shaped substrate used as a support for depositing thin layers of material. A core comprising a gas or consisting of a gas can be produced by the chemical or thermal decomposition of a solid substrate.

When the core is a porous substrate, when the core is a gas, or when the core comprises both a gas and a solid, the interference pigment of the invention can be obtained by depositing the layer of material comprising zinc sulfide on a solid support, which is subjected to a dissolution or thermal decomposition treatment to obtain a gaseous core or a core comprising gas.

In a first embodiment, the core of the pigment of the invention is a solid substrate. This substrate can be in different forms; for example, it is in the form of platelets or beads. In the case where the substrate is platelet-based, the dimension of the substrate will be, for example, the average length of a population of platelets, the length being the largest dimension of the platelets. The substrate can also be defined by its thickness, the thickness being able to represent the average thickness of said population.

The solid substrate can be transparent or semi-transparent to obtain a mother-of-pearl, or opaque to obtain an interference pigment with a metal effect.

In a particular embodiment, a transparent or semi-transparent substrate will be used.

The solid substrate may advantageously be selected from natural micas, synthetic micas, alkaline earth carbonates such as calcium carbonate, alkaline earth sulfates such as barium sulfate, natural pearls such as guanine or hypoxanthine, an alumina, aluminum, a silica, a borosilicate, a perlite, an organic polymer (such as a plastic), and a metal oxide such as zinc oxide or bismuth oxychloride. The substrate may consist essentially of one or more of these materials, without excluding the possible presence of impurities.

The core of the interference pigment of the invention is preferably free of zinc sulfide. It may nevertheless comprise zinc sulfide, as an impurity, for example an amount less than 0.1% by mass of the mass of the core.

In this first embodiment, the pigment of the invention may be a multi-layer interference pigment comprising a transparent or semi-transparent platelet substrate covered by at least one stack comprising at least:
a first layer of a material comprising zinc sulfide having a refractive index ranging from 2.30 to 2.90,
a second layer, contiguous to the first, of a material having a refractive index ranging from 1.00 to 2.10, and
a third layer of a material comprising zinc sulfide, contiguous to the second, having a refractive index ranging from 2.30 to 2.90, the difference in refractive index between two contiguous layers being greater than or equal to 0.3 and preferably ranging from 0.3 to 1.5, and the pigment being devoid of a layer containing titanium dioxide.

The platelet substrate is covered by at least one stack, in the sense that it can be covered with one or more stacks, each stack being able to comprise, in addition to the first layer, the second layer and the third layer, additional layers of other materials.

In a particular stack, the third layer is in contact and covers the second layer, while the second layer is in contact and covers the first layer.

The characteristics which have been described above in the context of the description of the pigment of the invention apply to the first variant embodiment.

The first layer can be colorless, in the sense that it does not absorb wavelengths in the visible range. The second layer can also be colorless.

In a second embodiment of the invention, the core of the pigment of the invention may comprise a gas or consist of a gas such as air, so that the pigment is in the form of particles called “core-shell” particles, the core of which is hollow and the shell comprises at least one interference layer of a material comprising zinc sulfide. As explained previously, such a core may be obtained from a solid support which is partially or totally removed by physical or chemical means, after the application of the shell consisting of the layers applied to the support.

According to this variant, the pigment of the invention is an interference single-layer or multi-layer pigment, comprising a platelet-shaped core covered with at least a first layer of a material comprising zinc sulfide, the support having a refractive index ranging from 1.00 to 2.10, characterized in that the support comprises a gas or consists of a gas such as air.

The first layer can have an average thickness ranging from 10 nm to 350 nm, the value of the average thickness being able to be equal to one of the values defined above.

The support advantageously has a refractive index ranging from 1.00 to 1.40. It can be in the form of a capsule of a solid material other than zinc sulfide. The term “capsule” means an object whose internal part is hollow or porous and whose outer part is a layer of a solid material. The material is preferably selected from transparent or semi-transparent materials resistant to acid and/or base attacks, such as for example SiO₂, Al₂O₃, ZrO₂.

In a particular embodiment, the core is a silica capsule. The silica layer has, for example, a thickness ranging from 1 nm to 100 nm.

The capsule may have a larger dimension ranging from 1 micron to 1000 microns.

The first layer of a material comprising zinc sulfide may cover the surface of the capsule, in the sense that it is in contact with the layer of solid material, and covers the entire surface thereof.

In another embodiment, the core is composed of a gas, and the pigment comprises a gaseous core, which may be delimited by the first layer of a material comprising zinc sulfide or by a layer of another material having a high-refractive-index.

The pigment of the second variant of the invention may be a single-layer pigment comprising a single layer of a material comprising zinc sulfide, or a multi-layer pigment comprising the first layer of a material comprising zinc sulfide, at least one layer of a material having a refractive index ranging from 1.00 to 2.10, and at least one second layer of a material comprising zinc sulfide. The difference between the refractive index of the first layer of a material comprising zinc sulfide and the refractive index of the layer of a material having a refractive index ranging from 1.00 to 2.10 is preferably greater than or equal to 0.80.

The pigment is advantageously devoid of a layer containing titanium dioxide, this layer being able to be interferential or not. In a preferred embodiment, the pigment comprises titanium dioxide in an amount of less than 0.1% by mass of the mass of the pigment.

As mentioned above, the material comprising zinc sulfide may be a material consisting essentially of zinc sulfide, or a material consisting essentially of zinc sulfide and at least one metal ion selected from Fe²⁺, Cu²⁺, Mn²⁺, Ag⁺, Au³⁺, Eu³⁺, Al³⁺, Ce³⁺ and In³⁺. The layer of a material comprising zinc sulfide may further be covered on one of its surfaces with a continuous or semi-continuous layer of metal nanoparticles such as gold, silver or copper nanoparticles.

The pigment of the second variant of the invention may comprise an outer protective layer, which may be of organic or mineral nature, and of hydrophilic or hydrophobic nature. Examples of suitable outer protective layers have been described above.

A method for synthesizing the interference pigment of the second variant of the invention comprises
a first step of preparing an aqueous dispersion of core particles, the dispersion having a pH ranging from 2 to 8 and a temperature close to boiling, and
a second step of coating the core particles comprising the addition, to said aqueous dispersion, of an aqueous solution of a zinc salt, such as zinc nitrate, and of an aqueous solution of sodium sulfide, the pH of the reaction medium obtained being maintained between 2 and 7 to obtain a suspension of solid particles covered with the material comprising zinc sulfide.

The characteristics which have been given above to describe the second object of the invention can be applied to the method for synthesizing the pigment of the second variant of the invention.

A pigment with a gas in the core provides better color saturation and brightness compared to prior art pigments.

When the core is a capsule, in particular a silica capsule, the method of the invention may comprise a step of synthesizing the capsule. The synthesis of the capsule may comprise a step of coating an organic or inorganic support with a layer of a material to obtain a coated support, the coating step being followed by a step of treating the coated support aiming at removing all or part of the material which constitutes the support.

The step of treating the coated support can be carried out by placing it in the presence of an acid aqueous solution or a base aqueous solution.

The coated support can also or alternatively undergo heat treatment by raising the temperature.

In a first embodiment, a layer of an acid-insoluble metal oxide is deposited on the surface of an acid-soluble support, which is preferably in the form of platelet inorganic particles.

The support may be platelet-shaped and made of an inorganic material, and may comprise, for example, a material selected from magnesium hydroxide, copper hydroxide, iron hydroxide, zinc oxide, calcium sulfate (such as gypsum), phyllosilicate (such as mica), and borosilicate.

The metal oxide is selected for example from SiO₂, Al₂O₃, ZrO₂ and SnO₂. The metal oxide can be deposited on the support by any method known to the person skilled in the art.

The treatment of the coated support may comprise one or more steps of treatment in aqueous solution.

For example, the coated support may be placed in an acid aqueous solution so as to attack and/or dissolve the support completely or partially, without dissolving the metal oxide layer. The acid aqueous solution is preferably an aqueous solution of a mineral acid selected from hydrofluoric acid, phosphoric acid, hydrochloric acid, sulfuric acid, nitric acid and mixtures thereof.

The coated support can successively undergo one or more steps of treatment in an acid medium, washing to eliminate the ions resulting from the dissolution of the inorganic support followed optionally by treatment in a base medium.

The treatment in aqueous solution can be carried out at a temperature ranging from 20° C. to 100° C., for a duration ranging from 1 hour to 24 hours.

In a second embodiment, the pigment comprises a core consisting of a gas.

Such a pigment can be produced, for example, by depositing a layer of a metal oxide or a layer of zinc sulfide on micron-sized graphite platelets. The coated graphite platelets are then subjected to calcination to remove the graphite and obtain metal oxide capsules or zinc sulfide capsules, depending on the material deposited on the graphite particles.

A pigment comprising a gaseous core can also be obtained by depositing a layer of zinc sulfide on a substrate, which can be chemically solubilized, according to a capsule synthesis method as described previously.

Examples of pigments of the invention comprise the following stacks of layers, the slash denoting the separation between two different layers, and the parentheses denoting a single layer comprising several materials:
Core/ZnS
Core/ZnS/low index material/ZnS
Core/ZnS/low index material/ZnS, the stacking of the three ZnS/low index/ZnS layers can be repeated several times in succession
Core/ZnS doped with a metal ion
Core/SiO₂/ZnS
Core/SiO₂/ZnS/low index material/ZnS
Core/ZnS/high index material different from ZnS
Core/(ZnS+high index material different from ZnS)
Core/ZnS/ZnS doped with a metal ion
Core/(ZnS+ZnS doped with a metal ion)
Core/high index material different from ZnS/ZnS
Core/high index material different from ZnS/low index material/ZnS.

In these examples, the high-index material other than ZnS has a refractive index ranging from 2.30 to 2.90, and may, for example, be Fe₂O₃, FeTiO₃, Cr₂O₃, Fe₃O₄, and the low-index material preferably has a refractive index ranging from 1.00 to 1.60. The stacks described may be covered with a continuous or semi-continuous layer of metal nanoparticles, for example gold, copper or silver nanoparticles, but also with a non-interferential protective layer. In these examples, a silica protective layer may be applied to the last layer of the stack. The core may be a gas or a capsule whose outer part is a metal oxide, such as SiO₂.

According to a particular embodiment, the pigment of the invention is different from a pigment comprising zinc sulfide and a platelet glass substrate having an average thickness of less than 1 μm. More specifically, the pigment of the invention may be different from a pigment in which the glass substrate is made of ECR glass of composition: SiO₂ (63-70%), Al₂O₃ (3-6%), CaO (4-7%), MgO (1-4%), B₂O₃ (2-5%), Na₂O (9-12%), K₂O (0-3%), TiO₂ (0.1-4%), ZnO (1-5%).

The pigment of the invention is also preferably different from a pigment comprising a glass platelet substrate, a zinc sulfide layer and a semi-transparent metal layer, the glass comprising 65-75% by mass of silicon oxide; 2-9% by mass of aluminum oxide; 0.0-5% by mass of calcium oxide; 5-12% by mass of sodium oxide; 8-15% by mass of boron oxide; 0.1-5% by mass of titanium oxide; and 0.0-5% by mass of zirconium oxide.

Finally, the pigment of the invention may be different from a platelet reflective pigment provided with an internal support comprising zinc sulfide whose thickness ranges from 50 nm to 1000 nm and which comprises two main opposite surfaces and at least one lateral surface. In this reflective pigment, a metal outer layer having a thickness ranging from 10 nm to 150 nm partially covers the internal support, so that the lateral surface of the internal support is not covered by the metal outer layer. This metal outer layer is for example selected from aluminum, copper, silver, gold, platinum, palladium, nickel, cobalt, tin, niobium, chromium and titanium.

The interference pigment of the invention can be incorporated into various manufactured products such as food products, paints, inks, dyes, plastics and cosmetics.

In a particular embodiment, the interference pigment described above may be used in cosmetic products, such as cosmetic care or makeup products.

A third other object of the invention therefore relates to a cosmetic composition, in particular a cosmetic care or makeup composition, comprising the pigment as described above.

In a particularly advantageous embodiment, the cosmetic composition of the invention comprises, in addition to the interference pigment, less than 3% by mass of titanium dioxide, in particular less than 1% by mass of titanium dioxide. Titanium dioxide may be provided in the composition as a filler, dye or UV filter. Titanium dioxide may also be provided in the composition by means of an ingredient which contains it, such as a pigment for example.

The cosmetic composition of the invention is preferably free of titanium dioxide.

Makeup products include eyeshadows, nail polishes, eyeliners, lipsticks, eyebrow makeup products, liquid and compact foundations, compact powders, and loose powders. Skincare products include, for example, white or tinted skincare creams and lip balms.

These products can be liquid or solid, contain water or be anhydrous. They are, for example, in the form of aqueous gels, water-in-oil or oil-in-water emulsions.

The cosmetic products may comprise, in addition to the interference pigment of the invention, at least one cosmetic ingredient known to the person skilled in the art, selected in particular from solvents, oils, pigments different from the pigment of the invention, lakes, dyes, waxes, cosmetically active compounds, surfactants, UV filters, gelling agents and thickeners. The person skilled in the art will be able to choose the cosmetic ingredients on the basis of their general knowledge.

In a particular embodiment, the cosmetic composition comprises an oily phase in which the interference pigment is dispersed, which comprises an outer protective layer of a hydrophobic nature.

A list of particular objects of the invention is given below.
1. An interference single-layer or multi-layer pigment comprising a core whose refractive index ranges from 1.00 to 2.10, covered with at least one layer of a material comprising zinc sulfide, said layer having an average physical thickness ranging from 10 nm to 350 nm.
2. The interference pigment according to object 1, characterized in that the average physical thickness ranges from 20 nm to 340 nm, from 30 nm to 330 nm, from 40 nm to 320 nm, from 50 nm to 310 nm, from 60 nm to 300 nm, from 70 nm to 290 nm, from 80 nm to 280 nm, from 90 nm to 270 nm, from 100 nm to 260 nm or from 110 nm to 250 nm.
3. The interference pigment according to object 1 or 2, characterized in that the pigment has a dimension ranging from 1 micron to 2000 microns.
1bis. The interference single-layer or multi-layer pigment comprising a core delimited by at least one layer of a material comprising zinc sulfide, the zinc sulfide representing between 1% and 100% by mass of the mass of the pigment.
2bis. The interference pigment according to object 1bis, characterized in that the zinc sulfide represents between 1% and 70% by mass, from 15% to 65% by mass, from 20% to 60% by mass, from 25% to 55% by mass, from 30% to 50% by mass or from 35% to 45% by mass of the mass of the pigment.
3bis. The interference pigment according to object 1bis or 2bis, characterized in that the pigment has a dimension ranging from 1 micron to 2000 microns.
1ter. The interference single-layer or multi-layer pigment comprising a core delimited by at least one layer of a material comprising zinc sulfide, the pigment being devoid of an interference layer containing titanium dioxide.
2ter. The interference pigment according to object 1ter, characterized in that the pigment comprises titanium dioxide in an amount of less than 0.1% by mass of the mass of the pigment.
3ter. The interference pigment according to object 1ter or 2ter, characterized in that the core comprises less than 0.1% by mass of TiO2 relative to the mass of the core.
4. The interference pigment according to one of the preceding objects, characterized in that the material comprising zinc sulfide is a material essentially consisting of zinc sulfide, or a material essentially consisting of zinc sulfide and at least one metal ion selected from Fe²⁺, Cu₂₊, Mn²⁺, Ag⁺, Au³⁺, Eu³⁺, Al³⁺, Ce³⁺ and In³⁺.
5. The interference pigment according to one of the preceding objects, characterized in that the layer of a material comprising zinc sulfide has a refractive index ranging from 2.30 to 2.90, preferably close to 2.40.
6. The interference pigment according to one of the preceding objects, characterized in that the core is a gas or a solid substrate.
7. The interference pigment according to one of the preceding objects, characterized in that the solid substrate is selected from natural micas, synthetic micas, alkaline earth carbonates, alkaline earth sulfates, natural pearls such as guanine or hypoxanthine, an alumina, aluminum, a silica, a borosilicate, a perlite, an organic polymer, and a metal oxide.
8. The interference pigment according to one of the preceding objects, characterized in that the pigment is a single-layer pigment comprising a single layer of a material comprising zinc sulfide.
9. The interference pigment according to one of the preceding objects, characterized in that the pigment is multi-layered and comprises at least one alternation of a first layer of a material comprising zinc sulfide having a refractive index ranging from 2.30 to 2.90, and a second layer, contiguous to the first, of a material having a refractive index ranging from 1.00 to 2.10, the difference in refractive index between said first layer and said second contiguous layer being greater than or equal to 0.3, and preferably ranging from 0.3 to 1.5, more preferably being of the order of 1.3.
10. The interference pigment according to one of the preceding objects, characterized in that the layer of a material comprising zinc sulfide is covered on one of its surfaces with a continuous or semi-continuous layer of metal nanoparticles such as gold, silver or copper nanoparticles.
11. The pigment according to one of the preceding objects, characterized in that the pigment comprises an outer protective layer, which may be of organic or mineral nature, and of hydrophilic or hydrophobic nature.
12. A method for synthesizing a pigment, such as an interference pigment, which consists of the heterogeneous precipitation of a material comprising zinc sulfide onto solid particles.
13. The method for synthesizing a pigment according to the preceding object comprising:
   a first step of preparing an aqueous dispersion of solid particles, the dispersion having a pH ranging from 2 to 8 and a temperature close to boiling, and
   a second step of coating the solid particles comprising the addition, to said aqueous dispersion, of an aqueous solution of a zinc salt, such as zinc nitrate, and of an aqueous solution of sodium sulfide, the pH of the reaction medium obtained being maintained between 2 and 7 to obtain a suspension of solid particles covered with the material comprising zinc sulfide.
14. A cosmetic composition comprising an interference pigment according to one of objects 1 to 11.
15. The cosmetic composition according to the preceding object, characterized in that it comprises an oily phase in which the interference pigment is dispersed, which comprises an outer protective layer of a hydrophobic nature.

The invention is described in more detail by the following example. Unless otherwise stated, the temperature is comprised between 20° C. and 25° C., and the pressure is atmospheric pressure.

### Example 1: Synthesis of a Pigment Comprising a Layer of Zinc Sulfide

The equipment used comprises a titrator, a peristaltic pump and a thermoregulated reactor coupled with a stirring system.

### 1—Preparation of the Dispersion of Platelet Particles in the Reactor

An acid aqueous dispersion of mica particles (commercial reference of the company Topy industries, PDM-20L) at a concentration close to 10 g/L is heated with stirring to a temperature close to boiling.

### 2—Preparation of the Zn²⁺ Saline Solution

An acidified Zn²⁺ saline solution of 0.03M concentration is placed in a peristaltic pump to be injected at a flow rate of 0.5 mL/min into the reactor.

### 3—Titrating Solution and Titrator Conditions.

An Na₂S solution of 1 M concentration is prepared and constitutes the sulfur agent for the synthesis of the ZnS layer. The static acid pH set on the titrator is greater than 3.

### 4—Coating by Nucleation and Growth of Zinc Sulfide

Moderate stirring and a temperature close to boiling were applied throughout the synthesis during the addition of the saline solution into the reactor. The static stationary pH controlled by the previously chosen titrator is maintained throughout the synthesis by controlled addition of the dilute sodium sulfide solution. An addition of 100 mL of saline solution is used in this case.

### 5—Washing

Once the reaction was complete, the mica particles covered with zinc sulfide were centrifuged and redispersed in ethanol. They were then dried for 12 hours in an oven at 80° C.

### 6—Observation

A continuous and homogeneous layer of zinc sulfide nanoparticles is then observed on the mica particles at the end of the synthesis. The coverage rate as well as the thickness of the ZnS layer is controlled according to the volume of zinc salt introduced into the reaction medium. In this example, the addition of 100 mL of solution allows to obtain a white powder producing a yellow/orange reflection.

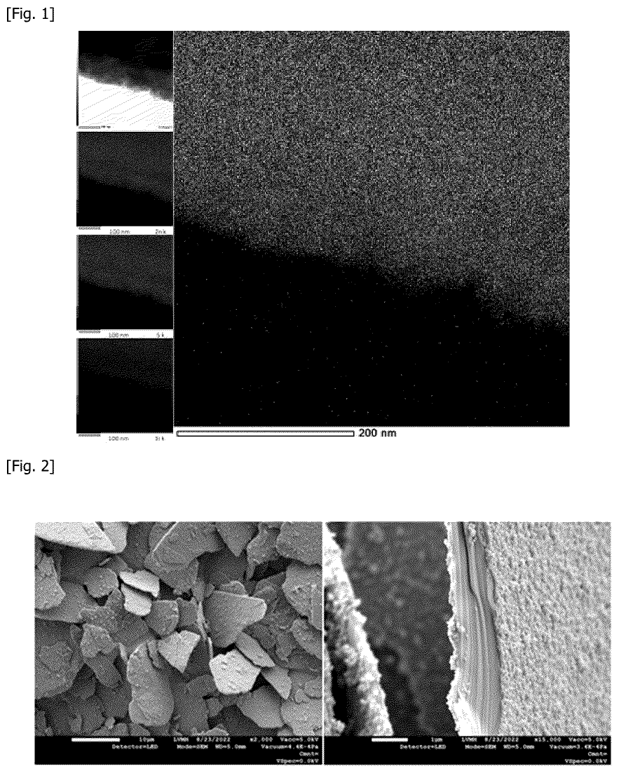

Microstructural analyses by transmission and scanning electron microscopy allowed to visualize the deposited thickness of the ZnS layer (FIG. 1) which is approximately 75 nm as well as its surface homogeneity (FIG. 2).

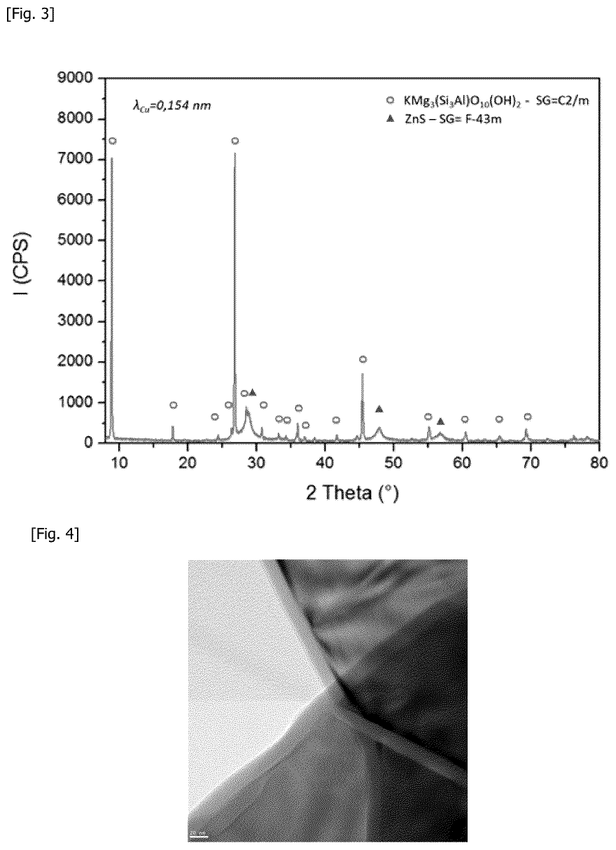

X-ray diffraction analyses allowed to show the crystallinity as well as the crystallographic nature of ZnS. (FIG. 3).

### Example 2: Interference Pigment Comprising a Hollow Core

### 1. Preparation of Gypsum Platelet Particles

A first solution consists of 41 g of anhydrous NaSO₄ salt dissolved in 1900 mL of distilled water. The solution is placed on a magnetic heating plate, stirred at 600 rpm and heated to 55° C.

A second solution is prepared by dissolving 42.4 g of CaCl₂·2H₂O in 100 mL of water containing 0.10 g of oxalic acid. This solution is stirred until homogeneous and poured all at once onto the first heated solution. Crystals form rapidly in solution.

The solution is stirred for 20 minutes at 50° C. The solution is filtered. The filtrate is clear. The solid formed is washed 3 times with water. All 3 washes are clear. The solid is dried in an oven at 75° C.

The obtained platelets were characterized by scanning electron microscopy (see FIG. 5). A heterogeneous distribution of gypsum particles in the form of more or less elongated platelets is observed.

2. Coating of Gypsum Platelet Particles with Silica

A first step of absorption of citrate ions on the gypsum plates is carried out. 1 g of gypsum powder is mixed with 30 mL of a 0.2 M citric acid solution at pH=4. After 10 min of stirring, separation by centrifugation and washing with water is carried out to remove excess citrate not adsorbed on the gypsum.

The pH is raised in a base medium by adding ammonia to the pellet with a few drops of water so as to be at a pH higher than 9 and to generate electrostatic repulsions between the particles.

A second step consists of the preparation of the reaction medium. A hydroalcoholic solution with a volume mixture of 24/76 (v/v) is prepared. Ammonia is added to the hydroalcoholic solution at a concentration of 0.37 M.

The base pellet containing 1 g of gypsum substrate is added to the hydroalcoholic solution.

A third step is to add the hydrolyzable precursor to the reaction medium. TEOS (tetraethyl orthosilicate), a hydrolyzable precursor of silica, is added all at once to the reaction medium. The reaction medium is sealed to prevent the ammonia from evaporating and kept stirring overnight.

A TEOS concentration of 0.00075 M is recommended for gypsum particles with a grain size of 5-25 microns to obtain a silica layer with a thickness of 20 nm. This thickness can be modulated depending on the amount of TEOS added.

At the end of this step, the powder is filtered then dried in an oven overnight at 75° C.

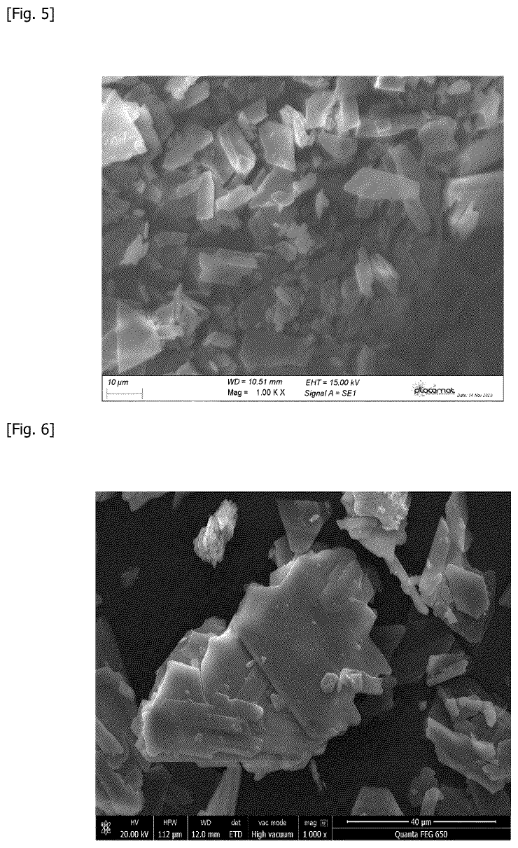

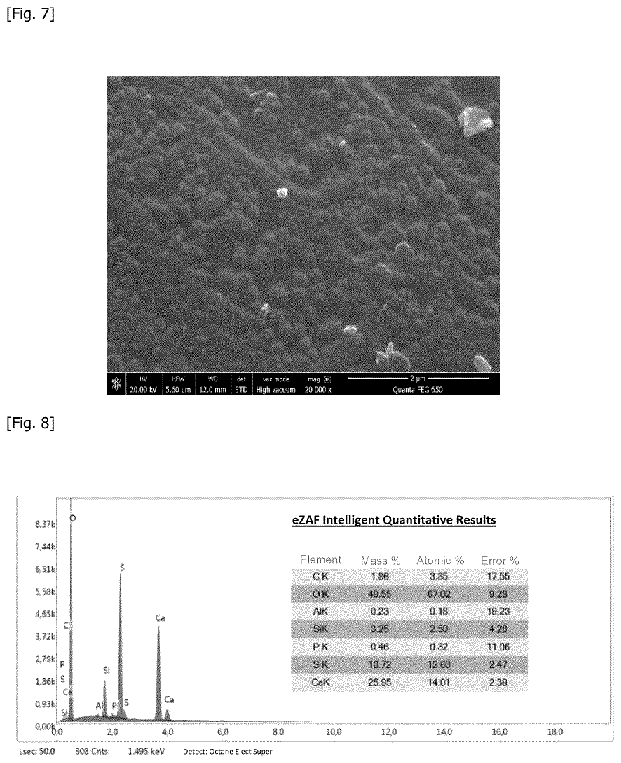

The characterization of encapsulated gypsum is presented in FIGS. 6 and 7.

The powder is dried in an oven at 75° C.

A characterization by EDX analysis was also carried out in order to verify the presence of silica characterized by the presence of Silicon (FIG. 8).

The surface microstructure is different from the smooth appearance previously obtained on gypsum boards, the surface roughness can be guessed. EDX analysis confirms the presence of silicon.

### 3. Deposition of a Layer of Zinc Sulfide

The obtained silica capsules are covered with a layer of zinc sulfide following the protocol of example 1.

### Example 3: Interference Pigment Comprising a Hollow Core

In a first step, 6.6 g of silica-encapsulated gypsum are introduced into a 5% nitric acid solution and the whole is heated under reflux for 3 hours.

The solution is centrifuged (10000 g—5 min) then washed with water and ethanol.

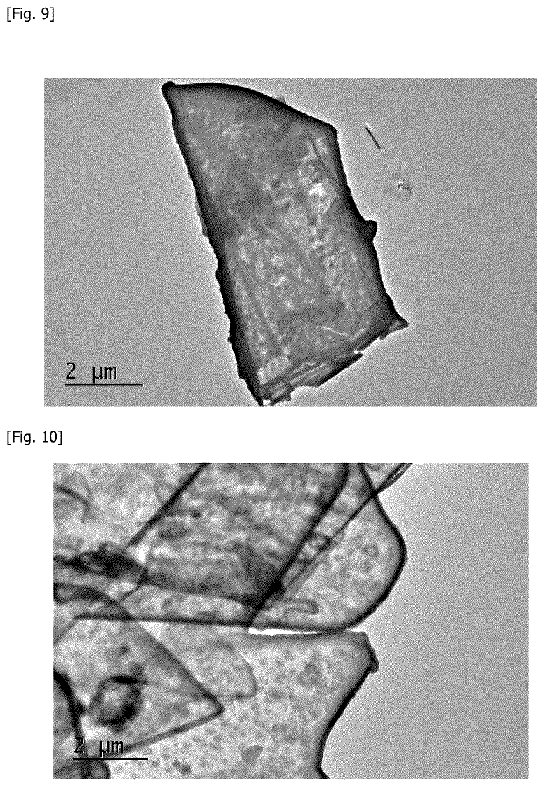

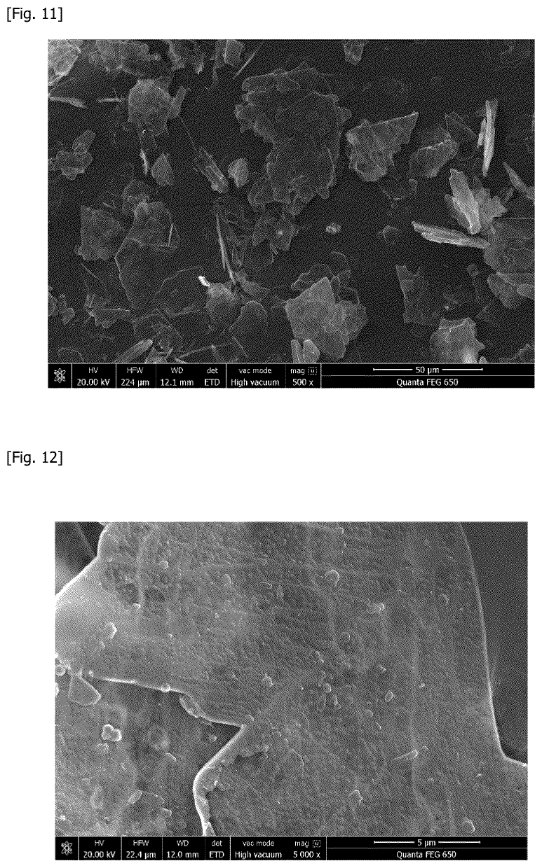

The powder is dried in an oven at 75° C. then gradually annealed in the oven (3° C./min up to 400° C., left for 1 hour, then lowered 3° C./min). The samples were characterized by transmission electron microscopy (FIGS. 9 and 10) and by scanning electron microscopy (FIGS. 11 and 12).

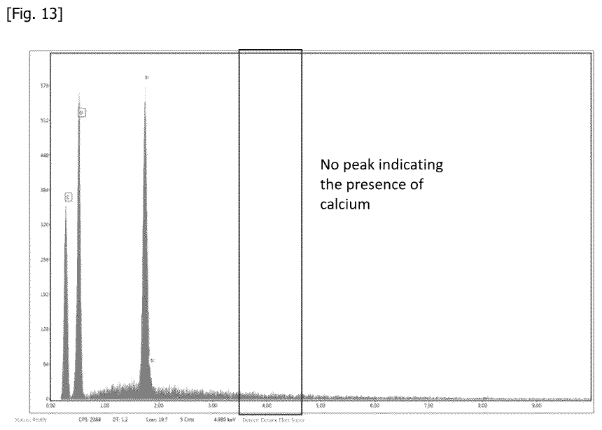

Electron microscopy characterizations show that more electrons pass through the particles during the acquisition of SEM images, resulting in more transparent platelets than before dissolution. The dissolution of the gypsum substrate is confirmed by EDX. The characteristic peak of Calcium at 3.6 eV (KαCa) justifying the presence of gypsum in FIG. 8 has completely disappeared after dissolution in FIG. 13. The peak of Silicon at 1.739 eV (KαSi) is in turn present. These coupled analyses clearly show that platelet particles with a hollow core have been obtained.

### Example 4: Synthesis of an Interference Pigment Comprising an Outer Protective Layer

Following the synthesis of the mother-of-pearl described in Example 1, the recovered dry powder will undergo various chemical treatments in order to be encapsulated by the silica.

A first step of absorption of citrate ions on the mother-of-pearl is carried out.
ZnS mother-of-pearl powder encapsulated on mica (base mica particle size 5-25 microns) is mixed with 30 mL of a 0.2M citric acid solution at pH=4. After 10 min of stirring, separation by centrifugation and washing with water is carried out to remove the excess citrate not adsorbed on the mother-of-pearl.
The pH is raised in a base medium by adding ammonia to the pellet with a few drops of water so as to be at a pH higher than 9 and to generate electrostatic repulsions between the particles.

A second step consists of the preparation of the reaction medium=
A hydroalcoholic solution with a volume mixture of 24/76 (v/v) is prepared. Ammonia is added to the hydroalcoholic solution at a concentration of 0.37 M.
The base pellet containing 1 g of mother-of-pearl substrate is added to the hydroalcoholic solution.

A third step consists of adding the hydrolyzable precursor to the reaction medium=
TEOS (tetraethyl orthosilicate), a hydrolyzable precursor of silica, is added all at once to the reaction medium. The reaction medium is sealed to prevent the ammonia from evaporating and kept stirring overnight.
A TEOS concentration of 0.00075 M is recommended in this example to obtain a silica layer with a thickness of 20 nm, which can be visualized on the transmission electron microscopy image given in FIG. 4. This thickness can be modulated depending on the amount of TEOS added.
At the end of this the powder is filtered then dried in an oven overnight to obtain a mica/ZnS/SiO₂ mother-of-pearl powder.

### Example 5: Cosmetic Formulas Containing a Pigment

Cosmetic formulas for makeup are prepared, particularly intended for application to the skin and/or lips. These formulas comprise an interference pigment according to the invention.

TABLE 1
Liquid lipstick

| INGREDIENTS | % BY MASS |
|---|---|
| MINERAL OIL | 5 |
| PASTY FAT | 10 |
| RED IRON OXIDES | 2 |
| ZINC OXIDE | 2 |
| ORGANIC LACQUERS | 2 |
| INTERFERENCE PIGMENT OF THE INVENTION | 2 |
| SILICA | 6 |
| MICA | 4 |
| ESTER OILS | QSP 100 |

TABLE 2
Anhydrous lip balm

| INGREDIENTS | % BY MASS |
|---|---|
| SILICA | 5 |
| POLYETHYLENE WAX | 5.5 |
| CANDELILLA WAX | 3 |
| SHEA BUTTER | 1.5 |
| IRON OXIDES | 5 |
| ORGANIC PIGMENTS (LAQUERS) | 2 |
| INTERFERENCE PIGMENT OF THE INVENTION | 3 |
| POLYDECENE HYDROGEN | QSP 100 |

TABLE 3
Lip balm

| INGREDIENTS | % BY MASS |
|---|---|
| NYLON | 8 |
| SILICA | 9 |
| POLYETHYLENE | 17 |
| GLYCERIN | 14 |
| INTERFERENCE PIGMENT OF THE INVENTION | 1 |
| ISONONYL ISONONANOATE | QSP 100 |

TABLE 4
Compact foundation powder

| INGREDIENTS | % BY MASS |
|---|---|
| MICA | 50 |
| SILICA | 10 |
| NYLON | 8 |
| MAGNESIUM STEARATE | 2 |
| SORBIC ACID | 0.1 |
| IRON OXIDES | 10 |
| INTERFERENCE PIGMENT OF THE INVENTION | 5 |
| GLYCOLS | 2 |
| ISONONYL ISONONANOATE | QSP 100 |
| CONSERVATIVES | QS |

TABLE 5
Emulsion foundation

| INGREDIENTS | % BY MASS |
|---|---|
| ESTER OILS | 6.5 |
| MINERAL OIL | 3.5 |
| CAPRYLIC/CAPRIC TRIGLYCERIDES | 2.2 |
| BEESWAX | 0.8 |
| METHYL POLYMETHACRYLATE | 1.1 |
| INTERFERENCE PIGMENT OF THE INVENTION | 3 |
| IRON OXIDES (black, red and yellow) | 10 |
| SILICA | 2 |
| WATER | QSP 100 |

TABLE 6
Anhydrous eyeshadow

| INGREDIENTS | % BY MASS |
|---|---|
| SILICA | 15 |
| SYNTHETIC FLUORPHLOGOPITE | 10 |
| IRON OXIDES | 8 |
| INTERFERENCE PIGMENT OF THE INVENTION | 15 |
| OILS AND WAXES | QSP 100 |

TABLE 7
Serum

| INGREDIENTS | % BY MASS |
|---|---|
| GLYCOL | 3 |
| GELLING POLYMER | 3 |
| MINERAL OIL | 2 |
| POLYETHYLENE GLYCOL | 1.5 |
| INTERFERENCE PIGMENT OF THE INVENTION | 4 |
| CONSERVATIVES | QS |
| PERFUME CONCENTRATE | 0.3 |
| WATER | QSP 100 |

TABLE 8
Scented Loose Powder

| INGREDIENTS | % BY MASS |
|---|---|
| MICA | 33.5 |
| SILICA (AND) LAUROYL LYSINE | 10 |
| INTERFERENCE PIGMENT OF THE INVENTION | 20.5 |
| CALCIUM ALUMINUM BOROSILICATE | 16.5 |
| CORN STARCH (AND) AQUA | 11 |
| CAPRYLYL GLYCOL | 1 |
| PENTYLENE GLYCOL | 1 |
| PRESERVATIVES AND PERFUMES | QS |

TABLE 9
Fluid care cream

| INGREDIENTS | % BY MASS |
|---|---|
| INTERFERENCE PIGMENT OF THE INVENTION | 0.5 |
| POLYURETHANE-35 | 4 |
| OILS | 12 |
| PASTY | 2 |
| FATTY ALCOHOL | 1.3 |
| STEARETH-21 | 1 |
| STEARETH-2 | 0.5 |
| UREA | 10 |
| WATER-BASED GELLING AGENT | 0.5 |
| PHENOXYETHANOL | 0.35 |
| ACRYLATES/C10-C30 ALKYL ACRYLATE | 0.3 |
| CROSSPOLYMER | |
| XANTHAN GUM | 0.1 |
| HYALURONIC ACID | 0.2 |
| POLYGLYCEROL | 17 |
| GLYCEROL | 3.9 |
| GLYCOLS | 3.6 |
| PRESERVATIVES AND PERFUMES | QS |
| WATER | QSP 100 |

1-15. (canceled)

16. A multi-layer interference pigment comprising a transparent or semi-transparent platelet substrate that is covered by at least one stack comprising at least:
one first layer of a material comprising zinc sulfide having a refractive index ranging from 2.30 to 2.90,
one second layer of a material having a refractive index ranging from 1.00 to 2.10, that is contiguous to the first layer, and
one third layer of a material comprising zinc sulfide having a refractive index ranging from 2.30 to 2.90 that is contiguous to the second layer,
the difference between the refractive indexes of two layers that are contiguous being greater than or equal to 0.3,
the pigment being devoid of one layer containing titanium dioxide.

17. The multi-layer interference pigment according to claim 16, wherein the multi-layer interference pigment comprises titanium dioxide in an amount of less than 0.1% by mass of the mass of the pigment.

18. The multi-layer interference pigment according to claim 16, wherein the third layer of a material comprising zinc sulfide and the third layer of a material comprising zinc sulfide have an average physical thickness ranging from 10 nm to 350 nm.

19. The multi-layer interference pigment according to claim 16, wherein the multi-layer interference pigment comprises an outer protective layer, which is preferably made of silica.

20. The multi-layer interference pigment according to claim 16, wherein the first layer is colorless.

21. The multi-layer interference pigment according to claim 16, wherein the second layer is colorless.

22. The multi-layer interference pigment according to claim 16, wherein the average physical thickness of the first layer and the average physical thickness of the third layer are independently from each other equal to a value ranging from 20 nm to 340 nm.

23. The multi-layer interference pigment according to claim 16, wherein the multi-layer interference pigment has a dimension ranging from 1 micron to 2000 microns.

24. The multi-layer interference pigment according to claim 16, wherein the material comprising zinc sulfide is a material essentially consisting of zinc sulfide.

25. The multi-layer interference pigment according to claim 16, wherein the material comprising zinc sulfide is a material essentially consisting of zinc sulfide and at least one metal ion selected from Fe²⁺, Cu²⁺, Mn²⁺, Ag⁺, Au³⁺, Eu³⁺, Al³⁺, Ce³⁺ and In³⁺.

26. The multi-layer interference pigment according to claim 16, wherein the transparent or semi-transparent platelet substrate is selected in the group consisting of natural micas, synthetic micas, alkaline earth carbonates, alkaline earth sulfates, natural pearls such as guanine or hypoxanthine, an alumina, a silica, a borosilicate, a perlite, an organic polymer, and a metal oxide.

27. The multi-layer interference pigment according to claim 16, wherein the first layer of a material comprising zinc sulfide or the third layer of a material comprising zinc sulfide is covered on one of its surfaces with a continuous or semi-continuous layer of metal nanoparticles such as gold, silver or copper nanoparticles.

28. A method for synthesizing a multi-layer interference pigment, comprising:
a first step of preparing an aqueous dispersion of solid particles, the dispersion having a pH ranging from 2 to 8 and a temperature close to the boiling point of the dispersion, and
a second step of coating the solid particles comprising adding an aqueous solution of a zinc salt and an aqueous solution of sodium sulfide to said aqueous dispersion, in order to form a reaction medium, wherein the pH of the reaction medium is maintained between 2 and 7 to obtain a suspension of solid particles that are covered with a material comprising zinc sulfide.

29. A cosmetic composition comprising an interference pigment according to claim 16.

30. The cosmetic composition according to claim 29, comprising less than 3% by mass of titanium dioxide, in particular less than 1% by mass of titanium dioxide.
